(19) United States
(12) Patent Application Publication
(10) Pub. No.: US 2017/0197829 A1
(43) Pub. Date: Jul. 13, 2017

(54) INCREASING CO/CO2 RATIO IN SYNGAS BY REVERSE WATER GAS SHIFT

(71) Applicant: Haldor Topsøe A/S, Kgs. Lyngby (DK)

(72) Inventor: Niels Ulrik Andersen, Gentofte (DK)

(73) Assignee: Haldor Topsøe A/S, Kgs. Lyngby (DK)

(21) Appl. No.: 15/313,053

(22) PCT Filed: May 27, 2015
(86) PCT No.: PCT/EP2015/061668

§ 371 Date: Nov. 21, 2016

(30) Foreign Application Priority Data
May 27, 2014 (DK) PA 2014 00286

(51) Int. Cl. C01B 3/38; C01B 3/32; C01B 3/36; F25J 3/06; B01D 53/22; B01D 53/047; B01D 53/14; F25J 3/02; B01J 19/24; C07C 29/151

(52) U.S. Cl. CPC C01B 3/38; B01J 19/245; C01B 3/32; C01B 3/36; C07C 29/151; B01D 53/22; B01D 53/047; B01D 53/1475; F25J 3/0252; F25J 3/0261; F25J 3/0655; F25J 3/0665; C01B 2203/0227; C01B 2203/025; C01B 2203/0475; C01B 2203/061; C01B 2203/1235; C01B 2203/0405; C01B 2203/046; C01B 2203/042; B01D 2256/22

(57) ABSTRACT

The present application relates to a production plant comprising—a synthesis gas generation step (1) arranged to receive a hydrocarbon or carboneous feedstock (2) and in a synthesis gas generation process provide a syngas, —a production step (4) arranged to receive the syngas and produce a product stream (5), —a reverse water gas shift step (4) arranged to receive a H2 rich gas stream (7) and a C02 feed (8) and in a RWGS step obtain a reverse shifted gas stream (9), and—means (10) for adding said reverse shifted gas stream (9) to the synthesis gas stream (3).

In revamps of existing plants the existing reactors, reformers etc. may put restraints on the possibilities for the updated process and/or plant. For example the catalyst volume in an existing plant may provide a limit for a process which means that the revamp cannot in an advantageous setup result in a need for an increased catalyst volume.

Thus in existing plants or other situations where constraints are made on reformers, reactors etc. there is a need for alternative processes and plants which increase efficiency without increasing the capacity needs above the available.

In a first object of the present process and plant is provided means for improving the amount and composition of a synthesis gas without increasing the need for reformer/reactor/catalyst volume.

These and other advantages are achieved by a production plant comprising
a synthesis gas generation step arranged to receive a hydrocarbon or carboneous feed stock and in a synthesis gas generation process provide a syngas
a production step arranged to receive the syngas and produce a product stream
a reverse water gas shift step arranged to receive a H₂ rich gas stream and a CO₂ feed and in a RWGS process obtain a reverse shifted gas stream, and
means for adding said reverse shifted gas stream to the synthesis gas stream whereby a plant which enable the production of a mixed synthesis gas stream having an improved CO/CO₂ ratio without resulting in an increase in the needed duty of the synthesis gas generation step for example comprising a reformer and/or an increased catalyst volume/heat transfer area in the production step.

The production step can be a methanol synthesis loop arranged to receive the syngas/reverse shifted gas mixture and produce a Methanol-rich product stream

The production step may also e.g. be a purification unit producing a product gas rich in Carbonmonoxide.

The synthesis gas generation step can in some advantageous embodiments be a reforming step, a gasification step, or a partial oxidation step depending on what feed is provided and/or on the production step. I.e. the synthesis generation step can be selected to provide an optimized inlet gas to the production step.

If the reverse shifted gas stream is provided downstream the synthesis gas generation step the synthesis gas generation step does not need to be dimensioned to receive the reverse shifted gas stream. This may be highly desirable in setups wherein volume/capacity of the synthesis gas generation step is an issue, which for example can be the case in revamps of existing plants.

If the RWGS step comprises a hydrogen recovery unit upstream the RWGS process the stream which enters the RWGS process has an increased H₂ ratio and a decreased content of other substances compared to the stream which enters the RWGS step.

Depending on the setup used the hydrogen recovery unit can be of different types such as a membrane unit, PSA unit or cryogenic unit.

From the recovery unit a residual gas stream may be provided e.g. to burners etc.

For example the H₂ rich gas stream can be a purge gas from the Methanol production loop. The purge gas can contain various substances which advantageously may be removed in which cases the purge gas can be passed through a hydrogen recovery unit as described above before it is fed to the RWGS process. Alternatively the H₂ rich stream may be sent directly to the RWGS step.

The CO₂ feed can be provided by various means. For example the CO₂ can be provided from underground natural CO₂ rich gas reservoir.

The CO₂ can also be provided from a purification unit (amine wash, PSA, etc.) removing CO₂ from a synthesis gas, flue gas, or natural gas depending on which sources of CO₂ are available or otherwise desirable in a given setup.

The RWGS step can be arranged in different ways with a range of suited catalysts. For example, the RWGS step may comprise a High Temperature Shift Catalyst (e.g. Topsøe SK201 or SK-501) or an UltraHigh Temperature Shift Catalyst for the RWGS process.

In setups where the production unit is a purification unit producing a CO stream or CO-rich stream the production unit may for example be a membrane unit or a cryogenic unit.

Also provided is a process for adjusting the CO/CO₂ ratio in a synthesis gas, said process comprising
in a production loop producing a product stream from a synthesis gas,
in an RWGS reactor producing a reverse shifted gas stream at least from a CO₂ feed and a H₂ rich gas stream, and
adding the produced reversed shifted gas stream to the synthesis gas upstream the production loop. I.e. in the present process a RWGS production step is used to provide a stream with an increased CO content, which stream with an increased CO content is added to the synthesis gas to obtain a mixed synthesis gas with a higher CO content thereby optimizing the production in the production loop.

The production loop can for example be a methanol production unit producing a methanol rich product stream or e.g. be a CO production/purification unit producing a CO rich stream.

If the H₂ rich gas stream is a purge gas from a Methanol loop a highly effective process is achieved wherein the off gas from the methanol production is used to optimize the composition of the syngas used in the methanol production.

In the process the RWGS shifted gas stream can advantageously be produced over a High Temperature Shift Catalyst (e.g. Topsøe SK-201 or SK-501) or a UltraHigh Temperature Shift Catalyst.

The RWGS inlet temperature can be in the range of 250 750° C. Often higher temperatures may be preferred as the RWGS conversion is favoured by higher temperatures. E.g. the inlet temperature can be 350° C. or above, such as 500° C. or above.

As the reverse water gas shift reaction is an endothermic reaction the outlet temperature in an adiabatic reactor will be lower than the inlet temperature, typically the temperature drop will be in the range 50-250° C., such as 60-125.

In several advantageous embodiments the reverse shift reaction converts 5-75% of the CO₂ into CO, resulting in a reverse shifted gas which has a CO/CO₂ ratio of 0.05-3, such as above 0.1 and/or below 2.

Generally the syngas may mainly comprise Hydrogen, Carbonmonoxide, Carbondioxide, Methane, and Water (small amounts of f.inst. Nitrogen, Argon, and Helium may also be present) In the case of methanol production the syngas may comprise

| | |
|---|---|
| H₂ | 65-75 vol-% |
| CO | 12-25 vol-% |
| CO₂ | 5-10 vol-% |
| CH₄ | 0-10 vol-% |
| H₂O | Saturated |

If the production step is a CO purification step the syngas generally comprises Hydrogen, Carbonmonoxide, Methane, Water, and Carbondioxide (small amounts of for example Nitrogen, Argon, and Helium may also be present) before the CO₂ removal step where the reverse shifted gas advantageous can be added

| | |
|---|---|
| H₂ | 50-70 vol-% |
| CO | 20-35 vol-% |
| CO₂ | 5-10 vol-% |
| CH₄ | 0-5 vol-% |
| H₂O | Saturated |

The H₂ rich gas stream may e.g. comprise Hydrogen, Carbonmonoxide, Carbondioxide, Water, and Methane. In case of a methanol loop purge gas the H₂ rich stream comprises

| | |
|---|---|
| H₂ | 70-85 vol-% |
| CO | 0-8 vol-% |
| CO₂ | 2-10 vol-% |
| CH₄ | 5-20 vol-% |
| Methanol | 0.3-1 vol-% |

The present process and plant may advantageously be part of a revamp of an existing plant such as a methanol production plant.

An example of parameters for the RWGS step is given below:

| | |
|---|---|
| Inlet | |
| RWGS | Outlet RWGS |

| | |
|---|---|
| Pressure, kg/cm² g | 25 |

| | | |
|---|---|---|
| Temperature, ° C. | 400 | 324 |
| Flow, Nm³/h | 45000 | 45000 |
| CO/CO₂ ratio | 0 | 0.259 |
| Gas Composition, mole-% | | |
| H₂ | 66 | 59 |
| CO₂ | 34 | 27 |
| CO | 0 | 7 |
| H₂O | 0 | 7 |

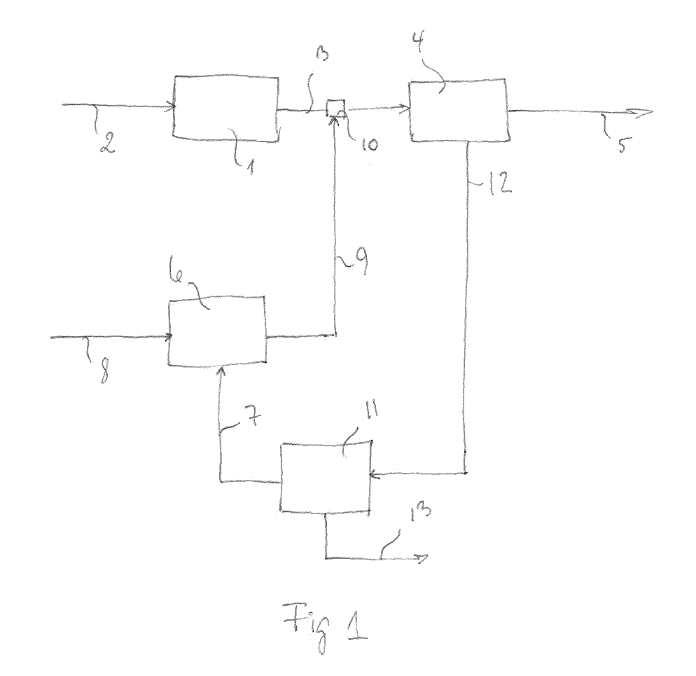

FIG. 1 shows a diagram of the plan/process according to the present invention wherein a synthesis gas generation step 1 is arranged to receive a hydrocarbon or carboneous feed stock 2 and in a synthesis gas generation process provide a syngas 3. A production step 4 is arranged to receive the syngas and produce a product stream (5). A reverse water gas shift step 6 is arranged to receive a H₂ rich gas stream 7 and a CO₂ feed 8 and in a RWGS process obtain a reverse shifted gas stream 9. The plant/process furthermore has means 10 for adding said reverse shifted gas stream to the synthesis gas stream. Upstream the reverse water gas shift process a H₂ recovery unit 11 can be arranged to provide a gas stream 7 which has an increased H₂ concentration compare to what is received from the production step 4. Such a H₂ recovery unit may for example be used where a purge 12 from the production step 4 is used to provide the H₂ rich stream. From the recovery unit a residual gas stream 13 may be provided e.g. to burners etc.

Thus according to the present invention is provided a process and a plant by which a mixture of CO₂ and H₂ stream is send to a reactor with a catalyst active towards the Water Gas Shift Reaction, a RWG shift (CO₂+H₂⇄CO+H₂O) can be obtained, improving the CO/CO₂ ratio, and thus the reactivity of the synthesis gas, reducing the required catalyst volume and/or heat transfer area in the production step, such as a methanol synthesis reactor. The present process and plant may be a particular advantage for revamp projects, where the size of reformer and/or Methanol reactor is given by existing structures.

1. Production plant comprising
a synthesis gas generation step arranged to receive a hydrocarbon or carboneous feed stock and in a synthesis gas generation process provide a syngas
a production step arranged to receive the syngas and produce a product stream
a reverse water gas shift step arranged to receive a H₂ rich gas stream and a CO₂ feed and in a RWGS step obtain a reverse shifted gas stream, and
means for adding said reverse shifted gas stream to the synthesis gas stream.

2. Production plant according to claim 1 wherein the production step is a methanol synthesis loop arranged to receive the syngas/reverse shifted gas mixture and produce a Methanol-rich product stream.

3. Production plant according to claim 1 wherein the production step is a purification unit producing a product gas rich in Carbonmonoxide.

4. Production plant according to claim 1, wherein the synthesis gas generation step is a reforming step, a gasification step, or a partial oxidation step.

5. Production plant according to claim 1, wherein the reverse shifted gas stream is provided downstream the synthesis gas generation step.

6. Production plant according to claim 1, wherein the RWGS step comprises a hydrogen recovery unit upstream the RWGS step.

7. Production plant according to claim 1, any of the proceeding claims wherein the H₂ rich gas stream is a purge gas 12 from the production step.

8. Production plant according to claim 1, wherein the CO₂ feed is provided from underground natural CO₂ rich gas reservoir.

9. Production plant according to claim 1, wherein the CO₂ feed is provided from a purification unit such as amine wash, PSA, etc. removing CO₂ from a synthesis gas, flue gas, or natural gas.

10. Production plant according to claim 1, wherein the RWGS step comprises a High Temperature Shift Catalyst (e.g. Topsøe SK-201 or SK-501) or a UltraHigh Temperature Shift Catalyst.

11. Production plant according to claim 1, wherein the hydrogen recovery unit is a membrane unit, PSA unit or cryogenic unit.

12. Production plant according to a claim 3 wherein the purification unit producing a CO stream or CO-rich stream is a membrane unit or a cryogenic unit.

13. A process for adjusting the CO/CO₂ ratio in a synthesis gas, said process comprising
in a Methanol loop producing a Methanol stream from a synthesis gas
in a RWGS loop producing in a RWGS step a shifted gas stream at least from a CO₂ feed and a H2 rich gas stream, and
adding the produced shifted gas stream to the syngas upstream the Methanol loop.

14. A process according to claim 13 wherein the H₂ rich gas stream is a purge gas from the Methanol loop.

15. A process according to claim 13, wherein the shifted gas stream is produced over a High Temperature Shift Catalyst (e.g. TopsøSK-201 or SK-501) or a UltraHigh Temperature Shift Catalyst.

16. A process according to claim 13, wherein the RWGS inlet temp is 250-750° C.

17. A process according to claim 13, wherein the RWGS outlet temp is 200-700° C.

18. A process according to claim 13, wherein the produced reverse shifted gas has a CO/CO₂ ratio of 0.05-3.
